(19) United States
(12) Patent Application Publication
(10) Pub. No.: US 2002/0081739 A1
(43) Pub. Date: Jun. 27, 2002

(54) Particle-mediated transformation of animal somatic cells

(76) Inventors: Winston J. Brill, Madison, WI (US); Dennis E. McCabe, Middleton, WI (US); Ning-Sun Yang, Verona, WI (US)

Correspondence Address: PowderJect Technologies Inc., 6511 Dumbarton Circle, Fremont, CA, 94555, US

(21) Appl. No.: 10/032,316

(22) Filed: Dec. 20, 2001

Related U.S. Application Data
(63) Continuation of application No. 08/352,263, filed on Dec. 7, 1994, now abandoned.
Application No. 08/352,263 is a continuation of application No. 08/148,029, filed on Nov. 5, 1993, now abandoned.
Application No. 08/148,029 is a continuation of application No. 07/494,933, filed on Mar. 14, 1990, now abandoned.
Application No. 07/494,933 is a continuation-in-part of application No. 07/371,869, filed on Jun. 26, 1989, now abandoned.

(51) Int. Cl. C12N 15/87; C12N 5/06

(52) U.S. Cl. 435/459; 435/320.1; 435/325

(57) ABSTRACT

A method is disclosed for the convenient transformation of the somatic cells of animals. Somatic cell transformation is useful for medical and veterinary care of genetic diseases, and other therapeutic or animal improvement purposes. The method makes use of an electric discharge particle acceleration apparatus which can inject very small particles of gold or other dense material carrying genetic constructs coated on them into the living cells of animals. The animals not only live, but there is no visible bruising or bleeding at the site of the treatment. The method is particularly adaptable since the force of the particle injection in such a spark discharge apparatus is adjustable by adjustments to the voltage of the spark discharge.

### FIELD OF THE INVENTION

The present invention relates to the technologies of genetic transformation in general and relates, in particular, to strategies for the genetic transformation of the non-germ line cells of animals.

### BACKGROUND OF THE INVENTION

Techniques have been developed for the genetic engineering of animals by which exogenous or foreign DNA can be inserted into the genomic DNA of animals. Typically in the prior art such genetic transformation of animals is performed by microinjection or by the use of retroviral based transformation vectors the effect of which is to genetically transform an animal embryo at a relatively early stage in development. The foreign DNA is incorporated into the genome of the animal embryo and then becomes incorporated into the genome of each of the daughter cells which arise from that embryo. Such genetic transformations insert the inserted DNA into all of the cells and the resulting whole organism including the germ line or sex cells of the organism. This insures that the genetic trait is passed to the progeny of the transformed animal in a normal Mendellian fashion.

There are occasions in which it would be desirable to transform animal cells in situ so that the animal can be imbued with the gene product of a genetic construction without affecting the genetic makeup of the germ line of the animal. In particular, for human applications the use of such somatic cell transformation avoids many of the ethical and philosophical problems which would arise from human intervention with the germ lines of human beings. The genetically engineered somatic cells offers the ability to make genetic corrections for inherited genetic disorders which consist of inactive or deleted enzymes necessary for normal biological functioning. It is also possible that such genetic transformations of somatic cells, and not germ line cells, may be desirable for certain therapeutic applications. For example, certain proteins offering therapeutic utility to patients must be currently injected into patients on a periodic strict time-line basis. However, the periodic injection of large quantities of proteins, even if done frequently, can result in an over supply of the protein shortly after an injection and a diminished supply shortly before the next injection resulting in potentially toxic shock following the injection and an insufficient supply for therapeutic efficacy just prior to the subsequent injection. An alternative strategy might be to engineer the gene for the desired protein into somatic cells of the animal or human so that the transformed cells would produce the therapeutic protein at a consistent level while they are live. By introducing the transforming gene into somatic cells which have a pre-defined and ascertainable life expectancy, such as skin cells for example, it is possible to create such an in vivo therapeutic production system which is time limited in the administration of the protein dosage to the animal or person being treated. In veterinary applications it may be desirable to introduce hormones or other growth factors or proteins for animal improvement, therapeutic, or disease inhibiting purposes into somatic cell portions of the animal which are not transient but which stay with the animal for its life expectancy.

While the vast majority of efforts directed at transformation of animal organisms or animal cells in culture have been directed toward the use of microinjection techniques or retroviral transformation vectors, the apparatus used for the transformation technique in accordance with the present invention is based on a quite different methodology of transforming the foreign DNA into the genome of the transformed somatic cells. There is one suggestion in the prior art of an apparatus containing some of the features which allow the apparatus of the present invention to be particularly adapted for its present use. As disclosed by Klein et al., Nature, 327: 70-73 (1987), an instrument for the acceleration of very small particles of metal carrying DNA thereon has been demonstrated to be effective for the transformation of plant cells in culture. The transforming DNA is coated onto very small particles which are physically accelerated by actually being shot on a ballistic projectile into the tissues to be transformed. While this apparatus has been demonstrated to have utility in transforming plant cells in culture, it suffers from a deficiency in that the adjustability of the force of impact of its particles is lacking making it a difficult apparatus to use for transformation of organisms over a wide range of kinetic energies of insertion of the particles into the transformed tissue.

### SUMMARY OF THE INVENTION

The prudent invention is directed toward a method of transforming the somatic cells of animals in vivo in which the exogenous DNA coding for the protein desired to be expressed in the somatic animal cells is coated onto small microparticles being of sufficiently small size so as to be able to enter the cells of animals without disrupting their biological function, placing an animal at a target site, and then accelerating the particles by means of an adjustable electric discharge so that the particles are accelerated at the target and into the cells of the target animal to thereby genetically transform a portion of the cells so treated so as to transform in vivo in the animal a number of cells to produce the protein coded by the exogenous gene.

It is a further object of the present invention to provide animals which have been treated with foreign DNA so that their somatic cells contain therein both an expressing exogenous gene construct and a very small particle of metallic material which carried the gene construct into the animal cell.

It is yet another object of the present invention to provide a method of transforming somatic skin cells of animals so that proteins are produced in the animals for limited time periods before the skin cells are shed in a normal biological fashion.

Other objects, advantages, and features of the present invention will become apparent from the following specification when taken in conjunction with the accompanying drawings.

### DESCRIPTION OF THE PREFERRED EMBODIMENT

The present invention is directed toward the transformation of the somatic cells of animals or human beings. By somatic cells as used herein it is meant to describe those cells of an animal or human being which when transformed do not change the genetic character or makeup of any of the germ or sex cells of the organism, so that when the animal or human reproduces through normal biological forms of reproduction, the introduced exogenous genetic material is not passed to the biological progeny of the organism.

The invention is directed toward the introduction of exogenous, often chimeric, genetic constructions into animal somatic cells. Such exogenous genetic constructions consist of DNA from another organism, whether of the same or different species, which is introduced into the transformed organism through human manipulation, by the artificial introduction of genes into the cells of the transformed organism. The exogenous DNA construction would normally include a coding sequence for a transcription product or a protein of interest, together with flanking regulatory sequences effective to cause the expression of the protein or the transcription product coded for by the coding sequence in the transformed cells of an organism. Examples of flanking regulatory sequences are a promoter sequence sufficient to initiate transcription and a terminator sequence sufficient to terminate the gene product, coded for by the gene, whether by termination of transcription or translation. Suitable transcriptional enhancers or enhancers of translational deficiency can be included in the exogenous gene construct to further assist the efficiency of the overall transformation process and expression of the protein result in the transformed cells. Other gene products than proteins may also be expressed by the inserted genetic construction. For example, the inserted construction could express a negative RNA strand effective either to suppress the expression of a native gene or to inhibit a disease pathology. The inserted construction could itself be RNA, as an alterative to DNA, if only transient expression of the gene product was desired.

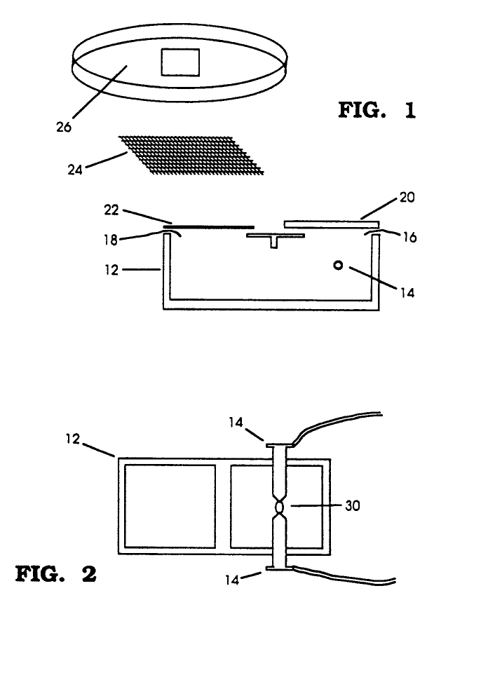

The present invention makes particular use of an apparatus for using an adjustable electric discharge to physically accelerate DNA coated onto small particles into the genetic material of somatic animal cells. A suitable apparatus for use within the present invention is illustrated in FIG. 1. The apparatus consists of a spark discharge chamber 12 into which are inserted two electrodes 14 which are spaced apart by a distance of approximately 1-2 mm. The spark discharge chamber is a horizontally extended rectangle having two openings 16 and 18 out its upward end. One opening 18 is covered by an access plate 20. The other opening, located opposite from the electrodes 14 is intended to be covered by a carrier sheet 22. The electrodes 14 are connected to a suitable adjustable source of electric discharge voltage. Such a source of electric discharge voltage would preferably include suitable electric switching connected to a capacitor of the 1 to 2 micro farad size range, with the amount of the voltage of the charge introduced into the capacitor being adjustable, such as through the use of an autotransformer, through a range of, for example, 1 to 50,000 volts. Suitable switching is provided so that the capacitor can be discharged through the electrodes 14 safely and conveniently by a user.

The carrier sheet 22 intended to be placed upon the opening 18 on the spark discharge chamber 12 is preferably a sheet of aluminized saran coated mylar. Above the opening in the discharge chamber, placed approximately 5-10 millimeters above it, is a retaining screen 24. Placed approximately 5-25 millimeters above the retaining screen is a target surface 26. In its use, the exogenous foreign gene construct intended to be transformed into the animal somatic cells is prepared by suitable DNA preparation techniques well known to one of ordinary skill in the art and it is dried onto small particles of a durable dense material such as gold, the particles typically being 1 to 3 microns in size. The carrier particles with the DNA dried thereon is then placed upon the carrier sheet 22 which is inserted on top of the spark discharge chamber 12. A target tissue, such as a live and anesthetized animal, is then placed adjacent to the target surface 26. Then a small droplet of water, approximately 2-4 microliters in size, is placed bridging between the ends of the electrodes 14. The access plate cover 20 is then placed over the top of the discharge chamber 12. At this point, the atmosphere between the carrier sheet 22 and the target is largely replaced with helium, by enclosing the apparatus and target and introducing helium in the enclosure in sufficient quantity to largely displace the atmospheric gases.

At this point the initiation of the spark discharge between the electrodes may be initiated by means of the use of the appropriate electronic switching. The force of the electric discharge bridges the spark discharge cap between the electrodes 14 instantly vaporizing the small droplet of water placed therebetween. The force of the vaporization of that water creates a shock wave within the spark discharge chamber 12 which radiates outward in all directions. The impact of the shock wave upon the carrier sheet 22 propels the carrier sheet 22 upwards with great velocity. The upwardly traveling carrier sheet 22 accelerates upward in direction until contacting the retaining screen 24. The presence of the helium provides less drag on the flight of the carrier sheet as well as less force for the shock wave to propagate to the target. At the retaining screen 24, the carrier sheet 22 is retained, and the DNA-coated particles previously applied thereto fly off of the carrier sheet and travel freely on toward the target surface. The particles therefor proceed into the target surface and enter the cells thereof. The momentum of the particles as they impact the surface of the target organism or tissue is adjustable based on the voltage of the initial electric discharge applied to the electrodes 14. Thus by variations in the amount of the electric energy discharged through the electrodes 14, the velocity by which the particles impact the target can be adjusted, and thus the depth of penetration of the particles into the tissue of a target, can be continuously adjusted over the range of adjustment of the electric discharge throughout the electrodes 14.

The apparatus of FIG. 1 has been previously demonstrated to be useful for the transformation of differentiated or undifferentiated tissue in a variety of forms including cellular masses in culture and whole growing organisms. It has been found through the work discussed herein that the apparatus is equally suitable for the transformation of either animal cells in culture or for the transformation of cells of animal somatic tissues. If cells are decided to be transformed, the cells can be placed upon a petrie plate or other media which can be inverted and used as the target surface 26 in the apparatus of FIG. 1. It is also possible to transform portions of whole animals by anesthetizing the animal as appropriate for the species and type of animal and then placing the anesthetized animal over a hole cut in a planar surface which will act as the target surface. The portion of the animal exposed through the hole in the target surface 26 will therefore be the treated target tissue transformed by the transformation process.

### EXAMPLES

a) Vectors Used

The following examples make use of a pair of chimeric expression vectors constructed so as to express in animals the enzyme chloramphenicol acetyltransferase, which confers resistance to the antibiotic chloramphenicol. Both chimeric gene expression plasmids have been previously described and demonstrated to be effective in animal transfection studies. The plasmid pSV2cat was described by Gorman et al., Mol. Cell Biol., 2:1044-1051 (1982) and the expression vector pRSVcat was described by Walker et al., Nature, 306:557-561 (1983). The plasmid pSV2cat is a chimeric cat gene construction including the Simian virus 40 (SV40) early promoter, the chloramphenicol acetyltransferase coding region from the plasmid pBR322-Tn9, the SV40 t-antigen intron, and the SV40 early polyadenylation region carried in the pBR322 vector. The plasmid does not contain a complete SV40 viral genome and is not infectious. The plasmid pRSVcat is also a pBR322 base plasmid that includes a chimeric Rous Sarcoma virus (RSV) long terminal repeat and promoter fragment, the cat coding region from Tn9, an intron from the mouse beta-globulin gene and the polyadenylation region from the SV40 early transcription unit. This plasmid does also not contain a viral genome and is not infectious. A related plasmid also used is designated pRSVNPTII and includes the Rouse Sarcoma Virus promoter, the coding region for the neomycin phosphotransferase-II gene, coding for resistance to the antibiotics kanamycin and G418, and a polyadenylation region from SV40. This plasmid as well does not contain a viral genome and is not infectious.

b) Mammalian (Human) Cells in Culture

A cell line designated MCF-7, derived from human mammary epithelial cells, was obtained. The cell line was propagated in vitro by an RPMI growth medium supplemented with 10% fetal calf serum. Cells of the line were then plated onto cover glasses (2×2 cm²) in 35 mm cultures and grown to 80% confluency giving approximately 5×10⁴ cells per cover glass.

The monolayer of cells on cover plates were then transformed by electrical discharge particle-mediated transformation using the plasmid pRSVNPTII and the transformation apparatus of FIGS. 1 and 2. The DNA was coated on gold crystalline beads at a density of 0.5 micrograms DNA per milligram gold beads. The apparatus was operated with spark discharge levels of OKV (control-no particles accelerated), 6 KV and 8 KV.

After the transformation procedure, the cells were put back in culture medium and grown for two days under standard conditions, i.e. without selection. During this period, cell growth was observed by microscope and found to be normal.

After two days, trypsin was applied to the cell cultures to remove the cells from the cover plates and the cells were plated in T25 culture flasks. To this culture medium, G418 was added as a selection agent a concentration of 250 micrograms per milliliter. Between 50% and 70% of the trypsinized cells attached to the plastic substratum of the flasks and spread out on the surface within an hour, indicating that these cells were still viable. The cells were grown under selection for three weeks. Mortality of the majority of cells was observed within the first week.

At the end of three weeks, individual cell colonies of 50 to 5,000 cells in clusters were observed in the transformed cell cultures. Control cell cultures, which were not subjected to the transformation process, but were subjected to G418 selection, showed no live colonies. The transformed cultures were then maintained under selection for an additional three weeks after which the number of colonies was counted.

At the end of the six weeks of selection culture, the transformed colonies were trypsinized, pooled and plated in T25 cultures. Cells continued to grow under selection. Ten weeks after transformation, approximately 20 million cells were generated from each transformed culture and the stably transformed MCF-7 cells could then be continuously grown as a stock culture.

The result of the colony counts at six weeks were used to evaluate transformation frequency using the transformation conditions tested. The results were as follows:

| Condition of | Resistant colonies |
|---|---|
| Transformation | per 0.5 × 10⁵ cells |
| 0 (no transformation) | 0 |
| 6 KV | 32 |
| 8 KV | 26 |

This indicates a transformation frequency in the approximate range of 5.2 to 6.4×10⁻⁴ cells among the cells exposed to the transformation process.

c) Mammalian Somatic Cells In Vivo

Mice were anesthetized with chloroform. On each mouse, an area of approximately 1 cm on its side was shaved. The mouse was then placed on a petri dish having a window cut in it with the shaved patch over the window.

DNA of pRSVcat was then coated onto 1-3 micron gold particles at a rate of 0.1 microgram of DNA per milligram of gold. The DNA was applied to the gold by precipitation with 25 mM spermidine with 6% polyethylene glycol (m.w. 3,000) with the addition of CaCl₂ to a final concentration of 0.6 M. The DNA coated gold beads were then rinsed in a 100% ethanol and applied to the carrier sheet as an ethanolic suspension at a concentration of dried gold coated beads of 0.05 mg/cm² of the carrier sheet.

The petrie dish with the mouse was placed over the apparatus of FIGS. 1 and 2 as the target surface. Prior to the electric spark discharge, the area between the carrier sheet and the target was flushed with helium (4 liters/min) for 15 seconds to reduce atmospheric drag on the carrier sheet and any possible shock wave damage to the animal.

After the transformation event, the animals all appeared unharmed and they seemed to recover completely. No bruising or bleeding of any kind was observed. After 24 hours the mice were sacrificed and the skin patch was removed and assayed for cat activity. The assay was performed by testing for acetylation activity with a radio-labeled of C¹⁴. Radioactive decay of the acetylated product could then be used as a measure of transformed enzyme activity.

For the various electric discharge levels and controls used, the results are summarized in the following table.

| | | | Counts per |
|---|---|---|---|
| | Counts per | Total Protein | 50 Microgram |
| Conditions | 50 microliter | Microgram/ul | Protein |
| 12 KV voltage & | 16,686 | 4.4 | 3792 |
| 1 micron | | | |
| 16 KV voltage & | 6,281 | 5.6 | 1121 |
| 1 micron | | | |
| 12 KV voltage & | 15,937 | 5.6 | 2854 |
| 1 micron | | | |
| 12 KV voltage & | 14,969 | 3.5 | 4276 |
| 1 micron | | | |
| DNA + Kaolin | 123 | 4.3 | 28 |
| (DNA soak control) | | | |
| DNA + DMSO | 117 | 2.3 | 50 |
| (DNA soak control) | | | |
| No DNA (control) | 119 | 5.6 | 21 |

These results indicate cat activity of at least 100 times background levels. Thus a foreign gene was delivered and expressed in somatic cells without evidence of harm or damage to the animal.

d) Amphibian Somatic Cells In Vivo

A (Xenopus) frog was anesthetized by chilling to 4° C. The chilled frog was also placed over a window cut in a petri dish lid and placed in the transformation apparatus of FIGS. 1 and 2 in the same fashion as with the mice.

The conditions and procedure used for the mice were repeated for the frog except for the following. The DNA used was pSV2cat. The DNA coated gold beads were loaded onto the carrier sheet at a density of 0.1 mg/cm².

Again after the transformation process, the animal appeared entirely unharmed. Again no bruising or bleeding of the animal was detected. After 24 hours, the frog was sacrificed and the 1 cm² patch of skin transformed was removed and assayed for cat activity. The results are tabulated on the following table.

| | | | Counts per |
|---|---|---|---|
| | Counts per | Total Protein | 50 microgram |
| Conditions | 50 microliter | Microgram/ul | Protein |
| 12 KV (belly) | 13,149 | 2.1 | 6261 |
| 16 KV (back) | 17,570 | 4.0 | 4392 |
| Control (belly) | 153 | 1.4 | 109 |
| Control (back) | 145 | 4.1 | 32 |

Thus, in this example levels of cat activity were observed at least in excess of 50 times background. Thus delivery and expression of a foreign gene was achieved in somatic cells without any identifiable damage or injury to the animal.

e) Amphibian Somatic Cells In Vivo—Systemic Product

In a second experiment on Xenopus, one animal was transformed under similar conditions, as above, but twice on the same frog (16 KV on its back, 12 KV on its belly). In this case only 0.05 mg/cm² instead of 0.1 mg/cm DNA coated beads were used. The frog was sacrificed after 20 hours, and the transformed skin patches sampled. In addition, a portion of non-transformed skin (shielded at the time of blasting) was sampled for CAT activity. The results are summarized in the following table.

| | | Total Protein | Counts Per |
|---|---|---|---|
| Results | Counts/50 ul | mg/ul | 50 ul Protein |
| 12 KV (belly) | 2,085 | 7.5 | 278 |
| 16 KV (back) | 9,343 | 8.6 | 1,086 |
| Untreated skin | 1,301 | 5.1 | 255 |
| from elsewhere | | | |
| on the same toad | | | |

Total activity in the transformed skin patches was reduced due to the lower bead loading rate, but the non-transformed skin sample clearly shows at least a 2 fold elevation above a non-transformed animal's skin, as in the previous experiment, thus showing a systemic accumulation of the enzyme produced in the transformed skin patches.

The present invention is not to be limited to the particular embodiment or examples disclosed above, but embraces all such modified forms thereof as come within the scope of the following claims.

1. A method of genetically transforming the somatic cells of an animal comprising the steps of:
coating copies of an exogenous genetic construction, the construction constructed so as to be able to express a gene product in the cells of the animal, onto carrier particles of dense material of a size very small in relation to the size of the animal cells;
layering the coated carrier particles onto a planar carrier sheet;
placing the carrier sheet onto a spark discharge chamber;
placing a droplet of water between the ends of a pair of spaced electrodes so as to bridge the gap between the electrodes;
placing the animal cells in the direction of travel of the carrier sheet;
initiating a discharge of high voltage electricity between the electrodes so that a spark bridges the gap between the electrodes, vaporizing the water droplet and accelerating the carrier sheet toward the animal cells, the carrier sheet being restrained from hitting the animal cells but the carrier particles traveling into the animals cells, the force with which the carrier particle hit the animal cells being adjustable by adjusting the voltage of the high voltage electricity applied to the electrodes so that the exogenous genetic construction is introduced into the animal cells with minimal damage to the cells.

2. The method of claim 1 wherein the exogenous genetic construction includes a protein coding DNA sequence and effective flanking regulatory sequences effective to express the protein in the animal cells.

3. The method of claim 1 wherein the exogenous genetic construction is a DNA sequence effective to express a negative RNA strand in the animal cells to inhibit a native gene or to inhibit disease processes.

4. The method of claim 1 wherein the animal cells are in culture out of the body of the animal.

5. The method of claim 1 wherein the animal cells are in vivo in the living animal and wherein the entire live animal is placed in the direction of travel of the carrier sheet.

6. The method of claim 5 wherein the animal cells that are transformed are in the skin of the animal.

7. The method of claim 1 wherein there is a retaining screen placed between the spark discharge chamber and the animal cells to retain the carrier sheet after it is accelerated toward the animal cells.

8. The method of claim 1 further comprising, before the step of initiating the discharge, the step of introducing helium gas into the area between the spark discharge chamber and the animal cells.

9. The method of claim 1 wherein the carrier particles are 1-3 micron gold particles.

10. Somatic cells of non-human animals transformed by the method of claim 1.

11. A non-human animal some of the somatic cells of which have been transformed by the method of claim 1.

12. A method of introducing an exogenous biological material into the somatic cells of a living animal comprising the steps of
coating copies of the exogenous biological material onto particles of a dense material small in size relative to the size of the cells of the animal;
placing the coated particles on an apparatus which is capable of accelerating the particles by means of the expansive force of a water droplet vaporized by the discharge of an electric potential through it; and
initiating the flow of the electric discharge through the apparatus to accelerate the coated carrier particles at and into the cells of the animal.

13. A method as claimed in claim 12 wherein the biological material is a genetic construction.

14. A method as claimed in claim 13 wherein the genetic construction is DNA.

15. A method as claimed in claim 13 wherein the genetic construction is RNA.

16. A non-human animal comprising cells containing particles of gold of a size small in relation to the size of the cells, and a chimeric genetic expression construction effective to cause expression of a gene product in those cells.
